(19) United States
(12) Patent Application Publication
(10) Pub. No.: US 2006/0058276 A1
(43) Pub. Date: Mar. 16, 2006

(54) Processes for the preparation and purification of rocuronium bromide

(76) Inventors: Oded Friedman, Talmey Yechiel (IL); Oded Arad, Rechovot (IL); Iosef Manascu, Omer (IL); Tamir Fizitzki, Beer-Sheva (IL); Boris Freger, Beer Sheva (IL); Joseph Kaspi, Givatayim (IL)

Correspondence Address: Martin MOYNIHAN;c/o ANTHONY CASTORINA, SUITE 207, 2001 JEFFERSON DAVIS HIGHWAY, ARLINGTON, VA, 22202, US

(21) Appl. No.: 11/180,716

(22) Filed: Jul. 14, 2005

Related U.S. Application Data
(60) Provisional application No. 60/587,900, filed on Jul. 15, 2004.
(60) Provisional application No. 60/587,901, filed on Jul. 15, 2004.

(51) Int. Cl. A61K 31/58

(52) U.S. Cl. 514/176

(57) ABSTRACT

Processes are provided herein for the preparation of pure rocuronium bromide and for the purification of impure rocuronium bromide obviating the need for column chromatography and that can be easily, conveniently and inexpensively scaled-up.

### RELATED PATENT APPLICATIONS

The present application claims the benefit of U.S. Provisional Application No. 60/587,900, filed Jul. 15, 2004, and U.S. Provisional Application No. 60/587,901, filed Jul. 15, 2004, the contents of which are herein incorporated by reference.

### FIELD AND BACKGROUND OF THE INVENTION

Neuromuscular blocking agents (e.g. tubocurarine chloride, pancuronium bromide, vecuronium bromide, rocuronium bromide, atracurium besylate) are known as compounds having the similar muscle paralyzing activity as the alkaloid curare or d-tubocurarine. Neuromuscular blocking agents interrupt transmission of nerve impulses at the skeletal neuromuscular junction and are typically divided into two types: competitive, stabilizing blockers (neuromuscular nondepolarizing agents) and noncompetitive, depolarizing agents (neuromuscular depolarizing agents). Both types prevent acetylcholine from triggering the muscle contraction and are typically used as anesthesia adjuvants in the operating theatre for aiding intubation i.e. relaxation of vocal cords, jaw muscles etc and also for surgery i.e. providing generalized muscle relaxation, as relaxants during electroshock, in convulsive states, etc. Typically, therapy is performed by i.v. administration of a suitable dosage form.

1-[(2β,3α,5α,16β,17β)-17-acetoxy-3-hydroxy-2-(4-morpholinyl)-androstan-16-yl-]-1-(2-propenyl)pyrrolidinium bromide, also known by the name rocuronium bromide, is a steroidal neuromuscular blocking agent having the structural formula (I):

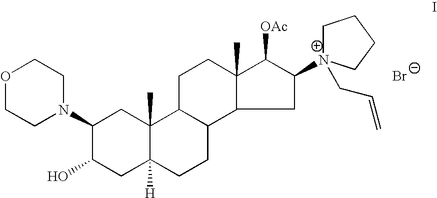

Rocuronium bromide and the intermediates thereof were first described in U.S. Pat. No. 4,894,369 to Sleigh et al., which is hereby incorporated by reference. In this U.S. patent the last step of the preparation of rocuronium bromide includes reacting 2-propenyl bromide with (2β,3α,5α,16β,17β)-2-(4-morpholinyl)-16(1-pyrrolidinyl)-androstane-3,17-diol 17-acetate, having the structure formula (II), in dichloromethane followed by column chromatography and precipitation of the pure product from a mixture of dichloromethane and diethyl ether.

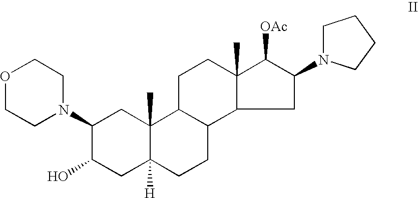

Hence the process described in the above-mentioned patent requires purification using column chromatography and therefore it provides relatively low yields of about 70-75% of the end-product.

Column chromatography is a complicated and expensive technique, which is inconvenient for industrial implementation, hence it is clear that this process cannot be advantageously used for large-scale production.

The above patent describes the physical and chemical characteristics of rocuronium bromide, but does not detail the identity and quantity of the impurities.

A paper by Zoltan et al. Current Medicinal Chemistry, 9(16), 1507-1536, 2002 deals with general synthesis, structure elucidation, pharmacological actions and structure-activity relationships studies of neuromuscular blocking agents used in the clinical practice and under others that are still under development. Rocuronium bromide is described in this paper in passing. No data was presented on its purification or purity degree.

A thorough patent and literature search failed to discover any alternative suitable processes for the preparation or for the purification of impure rocuronium bromide or references to its purity.

Thus, there is a need in the art for improved processes for the preparation and for the purification of rocuronium bromide that avoid the need of column chromatography and therefore can be easily, conveniently and inexpensively scaled-up.

Also, there is an unmet need for a process for manufacturing rocuronium bromide in high yield.

### SUMMARY OF THE INVENTION

In one aspect, the present invention relates to an improved process for the preparation of rocuronium bromide.

In another aspect, the present invention relates to an improved process for the preparation of pure rocuronium bromide.

In yet another aspect, the present invention relates to an improved process for the preparation of pure rocuronium bromide in high yield.

In another aspect, the present invention relates to improved process for the preparation of rocuronium bromide, obviating the need for column chromatography.

In another aspect, the present invention relates to an improved process for the preparation of pure rocuronium bromide substantially free of impurities.

In another aspect, the present invention relates to improved process for the preparation of pure rocuronium bromide that can be easily, conveniently and inexpensively scaled-up.

In another aspect, the present invention relates to an improved process for the preparation of pure rocuronium bromide comprising the steps of: 1. Reacting (2β,3α,5α,16β,17β)-2-(4-morpholinyl)-16(1-pyrrolidinyl)-androstane-3,17-diol, 17-acetate with an excess of allyl bromide in the presence of a suitable solvent; 2. Pouring the reaction mixture to a stirred anti-solvent; 3. Isolating the precipitated product in a pure form; and 4. Drying the product

By drying, in the context of this invention, it is meant removing the organic volatiles by one of the known in the art drying technologies including vacuum ovens, tray ovens, rotary ovens, and fluidized bed dryers.

By impure rocuronium bromide, in this context, it is meant a product having impurities such as starting materials, catalyst components and by-products in amounts, which are not in accordance with the allowed level for a pharmaceutical product with respect to the total weight of the product. Moreover, the impure rocuronium bromide as used herein refers to a rocuronium bromide isolated from any process conventionally known in the art or to be developed in the future

By substantially free, in this context, it is meant a product having impurities as defined hereinbefore in an amount; which is with accordance to pharmaceutically acceptable level.

Pure rocuronium bromide, in this context refers to a product containing rocuronium bromide and impurities in amount of less than about 0.5% w/w and preferably less than about 0.1% w/w, with respect to the total weight of the product.

The present invention deals also with processes for the purification of impure rocuronium bromide as defined hereinabove.

In yet another aspect, the present invention relates to an improved process for the purification of impure rocuronium bromide.

In another aspect, the present invention relates to improved process for the purification of impure rocuronium bromide in high yield.

In yet another aspect, the present invention relates to an improved process for the purification of impure rocuronium bromide obviating the need for column chromatography.

In another aspect, the present invention relates to improved process for the purification of impure rocuronium bromide characterized in that the product is substantially free of impurities.

In yet another aspect, the present invention relates to a process for obtaining a pure rocuronium bromide comprising the steps of: 1. Dissolving impure rocuronium bromide in a suitable solvent; 2. Pouring the reaction mixture to a stirred anti-solvent; 3. Isolating the wet precipitated product; and 4. Drying the product.

In yet another aspect, the present invention relates to a process for obtaining a pure rocuronium bromide comprising the steps of: 1. Dissolving impure rocuronium bromide in a suitable solvent; and 2. Drying, spray-drying or lyophilizing the product.

In yet another aspect, the present invention relates to a process for obtaining a pure rocuronium bromide comprising the steps of: 1. Suspending impure rocuronium bromide in a suitable anti-solvent; 2. Isolating the precipitated product in a pure form; and 3. Drying the product.

### DESCRIPTION OF THE PREFERRED EMBODIMENTS

The following detailed description is provided to aid those skilled in the art in practicing the present invention. Even so, this detailed description should not be construed to unduly limit the present invention as modifications and variations in the embodiments discussed herein can bc made by those of ordinary skill in the art without departing from the spirit or scope of the present inventive discovery.

The present invention meets a need in the art for improved processes for the preparation and for the purification of rocuronium bromide in high purity, in high yield and without the need for chromatographic purification.

In accordance with the present invention, an improved process for preparing a pure rocuronium bromide having the structure of Formula I in a high yield is provided. The process comprises 1. Reacting (2β,3α,5α,16β,17β)-2-(4-morpholinyl)-16(1-pyrrolidinyl)-androstane-3,17-diol, 17-acetate with an excess of allyl bromide in the presence of a suitable solvent; 2. Pouring the reaction mixture to a stirred anti-solvent; 3. Isolating the wet precipitated product in a pure form; and 4. Drying the product.

In one embodiment of the present invention, step (1) is carried out in the presence of an organic solvent.

In a preferred embodiment of the present invention, step (1) is carried out in an organic solvent.

As used herein, the term “solvent” refers to a single compound or a mixture of compounds. The term “organic solvent” means a solvent conventionally understood as such in the art, including a solvent in which non-polar or hydrophobic compounds are preferentially and substantially soluble.

Non limiting examples of organic solvents usable in context of the present invention include halogenated hydrocarbons, acetonitrile, N,N-dimethylformamide, N,N-dimethylacetamide, dimethylsulfoxide, and the like and mixtures thereof.

In preferred embodiment of the present invention, step (1) is carried out in halogenated hydrocarbons or in acetonitrile, more preferably in dichloromethane or in acetonitrile or in any mixture thereof.

In another embodiment of the present invention, step (1) is conducted at a temperature in the range of from about 10° C. to about 50° C., more preferably from about 15° C. to about 30° C., most preferably at an ambient temperature.

In another embodiment of the present invention, allyl bromide is added in an excess ranges from 5-fold to 30-fold relative to (2β,3α,5α,16β,17β)-2-(4-morpholinyl)-16(1-pyrrolidinyl)-androstane-3,17-diol, 17-acetate, more preferably from 10-fold to 20-fold, most preferably of about 17-fold.

In yet another embodiment of the present invention, the mixture of step (1) is poured into a stirred anti-solvent or cold anti-solvent in such a way so as to result in precipitation. The term “anti-solvent” is defined as any solvent in which the rocuronium bromide is poorly soluble.

Non-limiting examples of anti-solvents usable in context of the present invention include alkyl acetates, dialkyl ethers, wherein the dialkyl groups are the same or different, and low boiling point hydrocarbons or matures thereof.

As used herein, the term “alkyl” refers to a saturated aliphatic hydrocarbon including straight chain and branched chain groups. Preferably, the alkyl group has 1 to 10 carbon atoms. Whenever a numerical range; e.g., “1-10”, is stated herein, it means that the group, in this case the alkyl group, may contain 1 carbon atom, 2 carbon atoms, 3 carbon atoms, etc., up to and including 10 carbon atoms. More preferably, it is a medium size alkyl having 1 to 7 carbon atoms. Most preferably, it is a lower alkyl having 1 to 5 carbon atoms.

As used herein, the term “low boiling point hydrocarbons” refers to a saturated or unsaturated aliphatic hydrocarbon including straight chain and branched chain groups. Preferably, the hydrocarbon has 5 to 10 carbon atoms. Whenever a numerical range; e.g., “5-10”, is stated herein, it means that the hydrocarbon, may contain 5 carbon atoms, 6 carbon atoms, 7 carbon atoms, etc., up to and including 10 carbon atoms. Most preferably, it is a medium size hydrocarbon having 5 to 7 carbon atoms.

Representative examples of anti-solvents that are usable in the context of the present invention include, without limitation, methyl acetate, ethyl acetate, isopropyl acetate, isobutyl acetate, methyl t-butyl ether, diisopropyl ether, diethyl ether, pentane, hexane, heptanes, petroleum ethers and mixtures thereof.

In yet another embodiment of the present invention, the product is isolated from the mixture from step (2) by filtration or centrifugation. The product thus obtained may be treated with a washing solution containing an anti-solvent as defined above.

In yet another embodiment of the present invention, in step (4) the isolated product from step (3) can be dried using conventionally known methods to give pure rocuronium bromide substantially free of impurities but may contain pharmaceutically unacceptable levels of residual organic solvent(s).

The drying stage may be carried out by increasing the temperature or reducing the pressure or a combination of both. Non limiting examples of drying technologies or equipments usable in context of the present invention include vacuum ovens, tray ovens, rotary ovens and fluidized bed dryers.

By residual organic solvent(s), in this context, it is meant a solvent or solvents that are trapped in the solid product and are not completely removed from the product during the manufacturing process.

In yet another embodiment of the present invention, in order to reduce the levels of the residual organic solvent(s) an additional purification step is conducted.

Such additional purification may be carried out by repeating the dissolution, precipitation, isolation process as described hereinabove, or by dissolving the purified molecule in an organic or inorganic solvent(s), such as water and spray-drying or freeze-drying. These additional purification procedures result in pure rocuronium bromide comprising residual organic solvent(s) in an amount, which is with accordance to pharmaceutically acceptable level.

In another embodiment of the present invention, rocuronium bromide obtained by the process described hereinabove is further dried using conventional methods to give pure rocuronium bromide comprising residual organic solvent(s) in an amount, which is with accordance to pharmaceutically acceptable level, in high yield. Non-limiting examples of the additional drying methods usable in context of the present invention include spray-drying and freeze-drying.

In another embodiment of the present invention, rocuronium bromide obtained by the process described hereinabove is dissolved in a solvent and spray-dried or freeze-dried to give a pure rocuronium bromide comprising residual organic solvent(s) in amount, which is with accordance to pharmaceutically acceptable level The preferred solvent is selected from a group consisting dichloromethane, acetonitrile and water.

In yet another embodiment of the present invention, when the solvent is water the process is preferably conducted in the dark and with the absence of oxygen.

In another embodiment of the present invention, rocuronium bromide obtained by the process described hereinabove is suspended in a volatile anti-solvent, filtered and dried by a conventionally known method to give pure rocuronium bromide comprising residual organic solvent(s) in an amount, which is with accordance to pharmaceutically acceptable level, in high yield. Representative examples of anti-solvents that are usable in the context of this aspect of the present invention include, without limitation, diethyl ether, methyl acetate, ethyl acetate and isobutyl acetate.

The above preparation process results in pure rocuronium bromide further comprising impurities in amount of less than about 0.5% w/w and preferably less than about 0.1% w/w, with respect to the total weight of the product.

The yield of the process is an important feature of the invention. As described in the examples, rocuronium bromide can be obtained in a yield of over 90%, more preferably over 91%, more preferably over 92%, more preferably over 93%, more preferably over 94%, more preferably over 95%, more preferably over 96%, more preferably over 97%, more preferably over 98%, more preferably over 99% and most preferably quantitatively with respect to the starting amount of the molecule having the structure formula (II).

The above preparation process results in pure rocuronium bromide containing residual organic solvent(s) in an amount, which is with accordance to pharmaceutically acceptable level, hence the product is suitable as a raw material for producing rocuronium bromide injections.

In accordance with the present invention, the first improved purification process for obtaining a pure rocuronium bromide in high yield comprises: 1. Dissolving impure rocuronium bromide in a suitable solvent; 2. Pouring thc mixture to a stirred anti-solvent; 3. Isolating the wet precipitated product; and 4. Drying the product.

In one embodiment of the present invention, step (1) is carried out in an organic solvent.

In preferred embodiment of the present invention, step (1) is carried out in halogenated hydrocarbons or in acetonitrile, more preferably in dichloromethane or in acetonitrile or in mixtures thereof.

In another embodiment of the present invention, step (1) is conducted at a temperature in the range of from about 10° C. to about 50° C., more preferably about 15° C. to about 30° C., most preferably at an ambient temperature.

In yet another embodiment of the present invention, the solution of impure rocuronium bromide from step (1) is poured into a stirred anti-solvent or cold anti-solvent in such a way so as to result in precipitation.

In yet another embodiment of the present invention, the purified product is isolated from the mixture from step (2) by filtration or centrifugation. The product thus obtained may be treated with a washing solution containing an anti-solvent as defined above.

In yet another embodiment of the present invention, the isolated product of step (3) can be dried in step (4) using conventionally known methods to give pure rocuronium bromide in high yield.

In accordance with the present invention, the second improved purification process for obtaining a pure rocuronium bromide in high yield comprises: 1. Dissolving impure rocuronium bromide in a suitable solvent; and 2. Drying, spray-drying or lyophilizing the product.

In yet another embodiment of the present invention, impure rocuronium bromide is dissolved in a solvent and spray-dried or freeze-dried to give a pure rocuronium bromide. The preferred solvent is selected from a group consisting of dichloromethane, acetonitrile and water.

In yet another embodiment of the present invention, when the solvent is water the process is preferably conducted in the dark and in the absence of oxygen.

In accordance with the present invention, the third improved purification process for obtaining a pure rocuronium bromide in high yield comprises: 1. Suspending impure rocuronium bromide in a suitable anti-solvent; 2. Isolating the precipitated product in a pure form; and 3. Drying the product.

In yet another embodiment of the present invention, impure rocuronium bromide is suspended in a volatile anti-solvent, filtered and dried by a conventionally known method to give pure rocuronium bromide in high yield. Representative examples of anti-solvents that are usable in the context of this aspect of the present invention include, without limitation, diethyl ether, methyl acetate, ethyl acetate, and isobutyl acetate.

In yet another embodiment of the present invention, the processes for the preparation and the purification of rocuronium bromide may be conveniently and inexpensively scaled-up.

The impure rocuronium bromide as used herein refers to a rocuronium bromide isolated from any process conventionally known in the art or to be developed in the future.

The above purification processes results in pure rocuronium bromide that is suitable as a raw material for producing rocuronium bromide injections.

Although, the following examples illustrate the practice of the present invention in some of its embodiments, the examples should not be construed as limiting the scope of the invention. Other embodiments will be apparent to one skilled in the art from consideration of the specification and examples. It is intended that the specification, including the examples, is considered exemplary only, with the scope and spirit of the invention being indicated by the claims which follow.

### EXAMPLES

Analysis of Rocuronium Bromide by High Performance Liquid Chromatography (HPLC):

High performance liquid chromatography (“HPLC”) was performed using the following conditions: Column and packing—Hypersil Silica 5 μ 250×4.6 mm, Thermo Hypersil-Keystone, P.N. 30005-254630; UV detection—UV operated at 210 nm; flow rate: 2 ml/min; Mobile phase: Buffer. Acetonitrile=1:9 (v/v); Buffer preparation: Weighing 4.53 g of Tetramethylammonium Hydroxide Pentahydrate into 1000 ml volumetric flask. Dissolving and completing the volume with water and adjusting the pH to 7.4 with 85% Phosphoric Acid; Injection volume: 5 μL; Run time: 2.5 times the retention time of rocuronium bromide.

Analysis of Rocuronium Bromide by Gas Chromatography (GC):

Instrument:

Agilent 6890 Series GC system, equipped with an FID detector and a split mode injector and PAL head space device.

Column:

DB-624, 30 m, ID=0.53 mm, film thickness 3 μm (J&W CN 125-1334 is suitable)

| Temperature Programming: | |
|---|---|
| Initial oven temperature: | 40° C. |
| Hold time: | 10 min. |
| Program 1 final oven temperature: | 130° C. |
| Heating rate: | 12° C./min. |
| Program 2 final oven temperature: | 250° C. |
| Heating rate: | 50° C./min |
| Final hold time | 6 min |
| Detector (FID) temperature: | 250° C. |
| Injector temperature: | 220° C. |
| Carrier gas: | Helium |
| Nominal initial flow: | 3.7 mL/min |
| Injector | |
| Split ratio 1:25 | |

| Conditions for head space injector: | |
|---|---|
| Injection volume: | 1000 μL |
| Incubation temperature: | 80° C. |
| Incubation time: | 10 minutes |
| Agitator speed: | 500 rpm |
| Syringe temperature: | 125° C. |
| Fill speed: | 300 μL/sec |
| Fill strokes | 0 |
| Pullup delay | 0 |
| Injection speed: | 1000 μL/sec |
| Post injection delay: | 200 ms |
| Syringe flushing time: | 300 sec |
| Cycle runtime: | 34 minutes |

Spray-Drying was Performed by:

Mini spray dryer model Buchi B-190 was used for spray drying. System description: Heater 1.8 KW, Temperature range: 40-220° C., Evaporation Rate: approx 1500 ml/hour.

Freeze-Drying was Performed by:

VirTis AdVantage single shelf freeze-dryer with shelf temperatures that ranges from −70° C. to +60° C., with process condenser temperatures of −85° C.

### EXAMPLE 1

Acetyl chloride (23.0 ml, 0.3234 mole) was added to a solution of (2β,3α,5α,16β,17β)-2-(4-morpholinyl)-16-(1-pyrrolidinyl)-androstane-3,17-diol (107 gram, 0.2395 mole) in dichloromethane (2 l). The reaction mixture was set aside for 24 hours at 23° C. A sample was analyzed by HPLC and showed the mixture to contain 0.3% of the starting material, 35.1% of compound II and 55.5% of the diacetate. Aqueous HCl solution (10.5%, 305 Ml) was added and the mixture was heated to reflux for 4 hours. After cooling to 2° C. the mixture was neutralized to pH 7.2 by adding sodium carbonate solution (5%, 5 l). The aqueous phase was removed. Analysis showed a composition of 1.9% of the starting material, 86.3% of compound II and 8.8% of the diacetate. The organic phase was washed twice with water (2×500 ml), dried (Na₂SO₄) and the solvent was removed. The product was obtained as yellow crystals (98.4 gram, 84% yield). Analysis of the product showed it contains 1.6% of the starting material, 94.1% of compound II and 2.1% of (2β,3α,5α,16β,17β)-3,17-diacetoxy-2-(4-morpholinyl)-16-(1-pyrrolidinyl)-androstane).

### EXAMPLE 2

The material of example 1 was crystallized from acetone to give compound II of 99.9% purity. The product contained 0.09% of the starting material. The diacetate product was not detected. Similar results were obtained by crystallization from acetonitrile.

### EXAMPLE 3

A mixture of (2β,3α,5α,16β,17β)-2-(4-morpholinyl)-16(1-pyrrolidinyl)-androstane-3,17-diol 17-acetate (10 grams), allyl bromide (30 ml) and acetonitrile (40 ml) was stirred at room temperature for 3 hours. The solution was gradually poured in to a vigorously stirred solution of isobutyl acetate (480 ml). The precipitated rocuronium bromide was filtered and dried,

Analysis of the product showed:
Total impurities (HPLC)—0.15%
Isobutyl acetate (GC)—5.7%
Acetonitrile (GC)—not detected

### EXAMPLE 4

Rocuronium bromide (0.5 gram) from example 3 was dissolved in degassed purified water (40 ml) and freeze-dried. The product was further oven dried at 40° C.

Analysis of the product showed:
Total impurities (HPLC)—0.33%
Isobutyl acetate (GC)—0.25%

### EXAMPLE 5

Rocuronium bromide (1.0 gram) from example 3 was dissolved in acetonitrile (2 ml). The solution was poured to ether (120 ml) with vigorous stirring. The product was filtered and dried at 40° C.

Analysis of the product showed:
Total impurities (HPLC)—0.03%
Isobutyl acetate (GC)—0.55%
Ether (GC)—1.5%
Allyl bromide (HPLC)—1 ppm

### EXAMPLE 6

A mixture of (2β,3β,5α,16β,17β)-2-(4-morpholinyl)-16(1-pyrrolidinyl)-androstane-3,17-diol 17-acetate (5 grams), allyl bromide (13 ml) and acetonitrile (20 ml) was stirred at room temperature for 3 hours. The solution was poured to ether (120 ml). The product was filtered and dried.

Analysis of the Product Showed:
Total impurities (HPLC)—0.35%
Ether (GC)—0.66%
Acetonitrile (GC)—0.17%

### EXAMPLE 7

Rocuronium bromide (1.5 gram), prepared in a similar manner as described in example 3, was dissolved in dichloromethane (50 ml). The solution was spray dried at 120° C.

Analysis of the product showed:
Total impurities (HPLC)—0.13%
Isobutyl acetate (GC)—3.8%
Dichloromethane (GC)—0.37%
Allyl bromide (HPLC)—3 ppm

### EXAMPLE 8

A mixture of (2β,3α,5α,16β,17β)-2-(4-morpholinyl)-16(1-pyrrolidinyl)-androstane-3,17-diol 17-acetate (10 grams), allyl bromide (26 ml) and acetonitrile (40 ml) was stirred at room temperature for 3 hours. The solution was gradually poured in to a vigorously stirred solution of isobutyl acetate (480 ml). The precipitated rocuronium bromide was filtered and dried.

Analysis of the product showed:
Total impurities (HPLC)—0.16%
Isobutyl acetate (GC)—2.0%
Acetonitrile (GC)—not detected

### EXAMPLE 9

Rocuronium bromide from example 9 (1 gram) was suspended with mixing in petroleum ether 60-80 (50 ml) for 24 hours with mixing. The product was filtered and oven dried.

Analysis of the product showed:
Total impurities (HPLC)—0.17%
Isobutyl acetate (GC)—0.13%
Petroleum ether (GC)—1.2%
Allyl bromide (HPLC)—7 ppm

### EXAMPLE 10

Rocuronium bromide from example 9 (1 gram) was suspended with mixing in methyl acetate (50 ml) for 24 hours with mixing. The product was filtered and oven dried

Analysis of the product showed:
Total impurities (HPLC)—1.0%
Isobutyl acetate (GC)—0.15%
Methyl acetate (GC)—0.38%
Allyl bromide (HPLC)—8 ppm

### EXAMPLE 11

Rocuronium bromide (0.5 gram) prepared according to example 7 was dissolved in degassed purified water (40 ml). The mixture was freeze-dried. The product was further oven dried.

Analysis of the product showed:
Total impurities (HPLC)—0.33%
Isobutyl acetate (GC)—0.25%

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A process for preparing a pure rocuronium bromide, the process comprising:
a. reacting (2β,3α,5α,16β,17β)-2-(4-morpholinyl)-16(1-pyrrolidinyl)-androstane-3,17-diol, 17-acetate with an excess of allyl bromide in the presence of a suitable solvent;
b. pouring the reaction mixture to a stirred anti-solvent;
c. isolating the wet precipitated product in a pure form; and
d. drying the product.

2. A process according to claim 1, wherein said organic solvent is selected from a group consisting of acetonitrile, N,N-dimethylformamide, N,N-dimethylacetamide, dimethylsulfoxide, and the like and mixtures thereof.

3. A process according to claim 2, wherein said organic solvent is dichloromethane or acetonitile or any mixture thereof.

4. A process according to claim 1, being conducted at a temperature in the range of from about 10° C. to about 50° C., more preferably from bout 15° C. to about 30° C., most preferably at an ambient temperature.

5. A process according to claim 1, wherein said allyl bromide is added in an excess ranges from 5-fold to 30-fold relative to (2β,3α,5α,16β,17β)-2-(4-morpholinyl)-16(1-pyrrolidinyl)-androstane-3,17-diol, 17-acetate, more preferably from 10-fold to 20-fold, most preferably of about 17-fold.

6. A process according to claim 1, wherein said anti-solvent is selected from a group consisting of methyl acetate, ethyl acetate, isopropyl acetate, isobutyl acetate, methyl t-butyl ether, diisopropyl ether, diethyl ether, pentane, hexane, heptanes, petroleum ethers and mixtures thereof.

7. A process according to claim 1, wherein said isolating is done by filtration or centrifugation.

8. A process according to claim 1, wherein said drying stage may be carried out by increasing the temperature or reducing the pressure or a combination of both.

9. A process according to claim 8, wherein said drying of the product is carried out by any of the technologies or equipments selected from a group consisting of vacuum ovens, tray ovens, rotary ovens and fluidized bed dryers.

10. A process according to claim 1, further comprising an additional purifying process.

11. A process according to claim 10, wherein said additional purifying process comprises:
e. suspending the product in an anti-solvent;
f. stirring the mixture; and
g. filtration and drying the mixture to obtain the pure product.

12. A process according to claim 10, wherein said additional purifying process comprises:
h. dissolving the product in a suitable solvent; and
i. drying the solution by spray drying or freeze drying to obtain the pure product.

13. A process according to claim 12, wherein said suitable solvent is selected from a group consisting of water, dichloromethane and acetonitrile and mixtures thereof.

14. A process according to claim 13, wherein said suitable solvent is water.

15. The process of claim 14, wherein said solvent is water said dissolving is conducted in the dark and with the absence of oxygen and said drying is freeze drying.

16. A process according to claim 13, wherein said suitable solvent is dichloromethane or acetonitrile, and said drying is preferably spray drying.

17. A process according to claim 1, wherein said pure rocuronium bromide contain impurities in amount of less than about 0.5% w/w and preferably less than about 0.1% w/w, with respect to the total weight of the product.

18. A process according to claim 1, wherein said pure rocuronium bromide further contain residual organic solvent(s) in an amount, which is with accordance to pharmaceutically acceptable level.

19. A process according to claim 1, wherein said pure rocuronium bromide is obtained in a yield of over 90%, more preferably over 91%, more preferably over 92%, more preferably over 93%, more preferably over 94%, more preferably over 95%, more preferably over 96%, more preferably over 97%, more preferably over 98%, more preferably over 99% and most preferably quantitatively with respect to the starting amount of the molecule having the structure formula (11).

20. A process according to claim 1, wherein said pure rocuronium bromide is suitable as a raw material for producing rocuronium bromide injections.

21. A process for obtaining a pure rocuronium bromide, the process comprising:
a dissolving impure rocuronium bromide in a suitable solvent;
b. pouring the reaction mixture to a stirred anti-solvent;
c. isolating the wet precipitated product; and
d. drying the product.

22. A process according to claim 21, wherein said organic solvent is selected from a group consisting of halogenated hydrocarbons and acetonitrile, more preferably dichloromethane and acetonitrile and mixtures thereof.

23. A process according to claim 21, being conducted at a temperature in the range of from about 10° C. to about 50° C., more preferably from about 15° C. to about 30° C., most preferably at an ambient temperature.

24. A process according to claim 21, wherein said anti-solvent is selected from a group consisting of methyl acetate, ethyl acetate, isopropyl acetate, isobutyl acetate, methyl t-butyl ether, diisopropyl ether, diethyl ether, pentane, hexane, heptanes, petroleum ethers and mixtures thereof.

25. A process according to claim 21, wherein said isolating is by filtration or centrifugation.

26. A process according to claim 21, wherein said drying step is done by using a technology selected from a group consisting of vacuum ovens, tray ovens, rotary ovens and fluidized bed dryers.

27. A process for obtaining a pure rocuronium bromide, the process comprising:
a dissolving impure rocuronium bromide in a suitable solvent; and
b. drying, spray-drying or lyophilizing the product, to thereby obtaining said pure rocuronium bromide.

28. A process according to claim 27, wherein said drying is by spray-drying or freeze-drying.

29. A process according to claim 27, wherein said suitable solvent is selected from a group consisting of water, dichloromethane and acetonitrile and mixtures thereof.

30. A process according to claim 29, wherein said suitable solvent is water.

31. The process according to claim 30, wherein said process is preferably conducted in the dark and with the absence of oxygen and said drying is freeze drying.

32. A process according to claim 30, wherein said suitable solvent is dichloromethane or acetonitrile and said drying is preferably by spray drying.

33. A process for obtaining a pure rocuronium bromide, said process comprising:
a. suspending impure rocuronium bromide in a suitable anti-solvent;
b. isolating the precipitated product in a pure form; and
c. drying the product.

34. A process according to claim 33, wherein said impure rocuronium bromide is suspended in a volatile anti-solvent.

35. A process according to claim 33, wherein said isolating is by filtration or centrifugation.

36. A process according to claim 33, wherein said drying step is done by using a technology selected from a group consisting of vacuum ovens, tray ovens, rotary ovens and fluidized bed dryers.

37. A process according to claims 19-36, wherein said pure rocuronium bromide further contain residual organic solvent(s) in an amount, which is with accordance to pharmaceutically acceptable level.

38. A pure rocuronium bromide, obtained without column chromatography, containing residual organic solvent(s) in an amount, which is with accordance to pharmaceutically acceptable level.

39. A pure rocuronium bromide, according to claim 38, containing impurities in amount of less than about 0.5% w/w and preferably less than about 0.1% w/w, with respect to the total weight of the product.

40. A pure rocuronium bromide according to claim 38, being suitable as a raw material for producing rocuronium bromide injections.
